(19) United States
(12) Patent Application Publication
(10) Pub. No.: US 2019/0240501 A1
(43) Pub. Date: Aug. 8, 2019

(54) LIGHT THERAPY TREATMENT APPARATUSES AND SYSTEMS, AND METHODS OF OPERATING THEREOF

(71) Applicant: 9369201 Canada Inc., Kitchener (CA)

(72) Inventors: Alexa Roeper, Kitchener (CA); Daniel Choi, Waterloo (CA); Adrian Carolli, London (CA); Aidan Hilborn, Toronto (CA)

(21) Appl. No.: 16/269,895

(22) Filed: Feb. 7, 2019

Related U.S. Application Data
(60) Provisional application No. 62/627,351, filed on Feb. 7, 2018.

(51) Int. Cl. A61N 5/06

(52) U.S. Cl. CPC A61N 5/062; A61B 2018/00642; A61N 2005/0626; A61N 2005/0652

(57) ABSTRACT

Various embodiments described herein generally relate to light therapy apparatuses and systems, and methods of operating the light therapy apparatuses. An example light therapy apparatus includes a main chamber with a controller; a first and second side chambers flexibly mounted to the main chamber; and a circuit board partially enclosed within the chambers. The circuit board includes a main board portion partially enclosed within the main chamber, the main board portion comprising a main array of light-emitting diodes (LEDs); a first board portion partially enclosed within the first side chamber, the first board portion comprising a first side array of LEDs; a second board portion partially enclosed within the second side chamber, the second board portion comprising a second side array of LEDs.

### FIELD

The described embodiments relate to light therapy apparatuses and systems, and methods of operating the light therapy apparatuses.

### BACKGROUND

When undergoing physiotherapy, patients are often assigned exercises to be completed outside physiotherapy sessions. Patients are also advised of activities that they should avoid. Unfortunately, many patients do not fully comply with these instructions from their physiotherapists. As a result, the recovery period tend to be prolonged or even possibly incomplete, which can increase the chances of future injuries.

### SUMMARY

The various embodiments described herein generally relate to light therapy apparatuses and systems, and methods of operating the light therapy apparatuses.

In accordance with an embodiment, there is provided a light therapy apparatus that includes a main chamber comprising a controller; a first side chamber flexibly mounted to the main chamber, the first side chamber including a first power supply; a second side chamber flexibly mounted to the main chamber, the second side chamber including a second power supply; and a circuit board partially enclosed within the main chamber, the first side chamber and the second side chamber, the circuit board including: a main board portion partially enclosed within the main chamber, the main board portion including a main array of light-emitting diodes (LEDs) and a controller mating member to engage with the controller; a first board portion partially enclosed within the first side chamber, the first board portion including a first side array of LEDs and a first power mating member to engage with the first power supply; a second board portion partially enclosed within the second side chamber, the second board portion including a second side array of LEDs and a second power mating member to engage with the second power supply; a first linking member connecting the first board portion to the main board portion; and a second linking member connecting the second board portion to the main controller portion.

In some embodiments, each of the main board portion, the first board portion and the second board portion has an array of openings corresponding to the respective main array of LEDs, the first side array of LEDs and the second side array of LEDs, and each of the main array of LEDs, the first side array of LEDs and the second side array of LEDs protrude through the respective array of openings.

In some embodiments, the first side chamber is mounted to the main chamber with a first flexible member; and the second side chamber is mounted to the main chamber with a second flexible member.

In some embodiments, the first flexible member at least partially shields the first linking member; and the second flexible member at least partially shields the second linking member.

In some embodiments, the light therapy system further includes a shielding layer partially enclosed within the main chamber, the first side chamber and the second side chamber, wherein the shielding layer at least partially shields the first linking member and the second linking member.

In some embodiments, the circuit board includes a flexible printed circuit board.

In some embodiments, the first side chamber is mounted to a first edge of the main chamber and the second side chamber is mounted to a second edge of the main chamber, the second edge being opposite to the first edge.

In some embodiments, the controller includes a LED driving component to control an operation of each of the main array of LEDs, the first side array of LEDs and the second side array of LEDs.

In some embodiments, each of the main array of LEDs, the first side array of LEDs and the second side array of LEDs comprises one or more LEDs with an operating wavelength in a range of approximately 800 nm to 940 nm.

In some embodiments, each of the main array of LEDs, the first side array of LEDs and the second side array of LEDs comprises approximately two LEDs for each surface area of 1 cm².

In accordance with an embodiment, there is provided a light therapy system comprising a light therapy apparatus disclosed herein; a computing device associated with the light therapy apparatus; and a therapy control server in communication with the light therapy apparatus and the computing device, the therapy control server including: a server controller operable to control a light therapy session at the light therapy apparatus; a communication component to transmit and receive operation signals to and from the computing device; and a memory to store operating data in respect of the operation of the light therapy apparatus.

In accordance with an embodiment, there is provided a method of operating a light therapy apparatus. The method including: receiving, from a computing device, an injury position signal identifying a location of an injury; operating a server controller remote to the computing device to define a light therapy treatment for the injury identified by the injury position signal, the light therapy treatment defining at least one of an intensity, a duration and a wavelength of an array of light emitting diodes (LEDs) at a light therapy apparatus; transmitting, by the server controller, a set of treatment parameters associated with the light therapy treatment to the computing device; and automatically initiating, by the computing device, the light therapy treatment at the light therapy apparatus associated with and remote from the computing device.

In some embodiments, the injury position signal includes a user identifier corresponding to a user with the injury and operating the server controller to define the light therapy treatment for the injury identified by the injury position signal includes: retrieving a user profile stored in a memory remote from the computing device for the user; and defining the light therapy treatment based at least on a treatment history associated with the user profile.

In some embodiments, defining the light therapy treatment based at least on the treatment history associated with the user profile includes identifying a prior injury similar to the injury; and defining the light therapy treatment for the injury based on a recovery progress of the prior injury.

In some embodiments, defining the light therapy treatment based at least on the treatment history associated with the user profile includes: identifying an ongoing treatment for the injury; determining a recovery progress of the injury based on the ongoing treatment; and adjusting the ongoing treatment for the injury based on the recovery progress of the injury.

In some embodiments, determining the recovery progress of the injury based on the ongoing treatment includes: generating a progress assessment interface at the computing device requesting an assessment of the recovery progress from the user; and receiving a recovery assessment signal from the computing device representing the recovery progress of the injury.

In some embodiments, the method includes: prior to initiating the light therapy treatment at the light therapy apparatus, operating the computing device to detect a power supply level at the light therapy apparatus; and in response to determining the power supply level is below a minimum power threshold, transmitting a power deficient notification to the computing device, otherwise, initiating the light therapy treatment at the light therapy apparatus.

Also provided is a device and mobile application which collects patient data feedback to vary the treatment parameters that the device delivers. The data collected includes any one or more of a selection of patient collected data. The patient collected data includes the location and originating tissue of the pain. Where the patient straps the device to a specific location on the body and extends and flexes the joint, the patient collected data includes a range of motion measurement. The patient collected data includes a self-reported pain measurement. The mobile application measures the change in self-reported pain since the last pain recording

Also provided is a back-end database which benchmarks the patient collected data against known parameters for a specific treatment. The specific treatment may be low-level laser therapy (LLLT). The back-end database may transmit the treatment parameters back to the mobile application which transmits it back to the device. The device receives the treatment parameters and adjusts the treatment program to the corresponding treatment settings

Provided herein is a method for tailoring treatments of therapeutic modalities where patient data is analyzed against a database of settings pertaining to the patient condition. The resulting parameters may be sent back to the treatment device via wireless transmission, including bluetooth, cellular data, or wifi.

Also provided herein is a method for improving the treatment settings database for therapeutic modalities using feedback from patient outcomes as recorded from a mobile app and/or device monitoring the patients recovery. The data used may include range of motion and pain levels.

### BRIEF DESCRIPTION OF THE DRAWINGS

Several embodiments will now be described in detail with reference to the drawings, in which:

FIG. 1 is a block diagram of an example light therapy system in accordance with an example embodiment;

FIG. 2 is an exploded view of some components of an example light therapy apparatus;

FIG. 3A is a perspective view of an inner surface of a top portion of the enclosure of FIG. 2 coupled with an example circuit board;

FIG. 3B is a perspective view of an outer surface of the top portion of FIG. 2A;

FIG. 4 is a rear perspective view of a bottom portion of an enclosure of another example light therapy apparatus;

FIG. 5 is a rear perspective view of an inner surface of a top main portion compatible with the bottom portion of FIG. 2;

FIG. 6 is a rear view of the inner surface of FIG. 5 coupled with an optional component;

FIG. 7 is a rear perspective view of an inner surface of a top first side portion compatible with the top main portion of FIG. 5;

FIG. 8 is a perspective view of a flexible member in accordance with an example embodiment;

FIG. 9 is a perspective view of an inner surface of a top portion of another example enclosure coupled with other components;

FIG. 10A is a front perspective view of an example controller;

FIG. 10B is a rear perspective view of the example controller of FIG. 10A;

FIG. 11 is a perspective view of an example circuit board;

FIG. 12A is a front view of another example light therapy apparatus with some components of a side chamber shown, in accordance with another example embodiment;

FIG. 12B is a side perspective view of the light therapy apparatus of FIG. 12A;

FIG. 12C is a partial rear view of the light therapy apparatus of FIG. 12A;

FIG. 13 shows an example chain of light therapy apparatuses;

FIG. 14 is a flowchart of an example embodiment of various methods of operating a light therapy apparatus;

FIG. 15A is a screenshot of an injury identification interface in accordance with an example embodiment;

FIG. 15B is a screenshot of a treatment initiation interface in accordance with an example embodiment;

FIG. 15C is a screenshot of a treatment termination interface in accordance with an example embodiment;

FIG. 16 is a screenshot of a treatment progress interface in accordance with an example embodiment; and

FIGS. 17A to 17H are screenshots of a treatment progress, in accordance with an embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

Light therapy treatment can be used to treat medical conditions related to the musculoskeletal system of a body of a patient. The patient can be a human or other animal. The injuries to the musculoskeletal system have been shown to have a faster recovery when light therapy is applied more frequently. In some cases, it is recommended that light therapy be applied daily or every other day.

The light therapy system described herein includes a light therapy apparatus that is portable. The light therapy apparatus can be in communication with a computing device, as will be described.

The portability of the light therapy apparatus facilitates application of the light therapy apparatus to the location of the injury. For example, in some cases, the light therapy apparatus can be worn by the user during treatment. The portability of the light therapy apparatus also allows a user to wear the light therapy apparatus for extended periods of time.

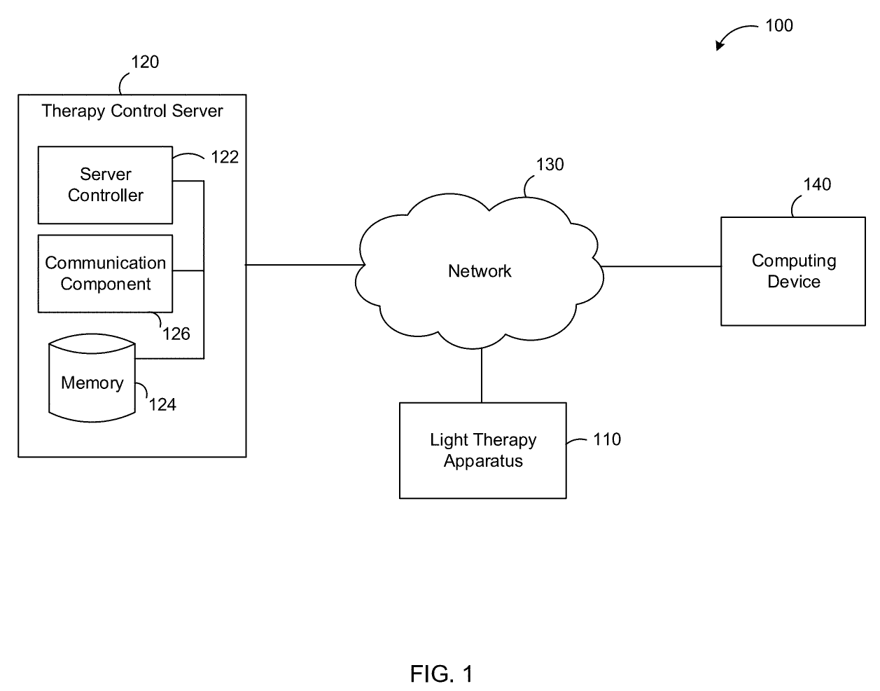

Reference is now made to FIG. 1, which illustrates a block diagram of an example light therapy system 100. The light therapy system 100 includes a light therapy apparatus 110, a therapy control server 120 and a computing device 140. The light therapy apparatus 110, the therapy control server 120 and the computing device 140 can be in communication via a network 130.

The therapy control server 120 includes a server controller 122, a communication component 126 and a memory 124. It will be understood that the therapy control server 120 may include one or more servers that may be distributed over a wide geographic area and connected via network. In some embodiments, each of the communication component 126, the memory 124 and the server controller 122 may be combined into a fewer number of components or may be separated into further components. The communication component 126, the memory 124 and the server controller 122 can be implemented in software or hardware, or a combination of software and hardware.

The server controller 122 can initiate a light therapy session at the light therapy apparatus 110 and can also define the parameters of the light therapy treatment. The server controller 122 can include any suitable processors, controllers or digital signal processors that can provide sufficient processing power depending on the configuration, purposes and requirements of the therapy control server 120. In some embodiments, the server controller 122 can include more than one processor with each processor being configured to perform different dedicated tasks.

The communication component 126 can include any interface that enables the therapy control server 120 to communicate with other devices and systems, such as the computing device 140 and the light therapy apparatus 110. In some embodiments, the communication component 126 can include at least one of a wireless network (e.g., Bluetooth™, Wi-Fi, WiMAX, etc.), Local Area Network (LAN), Ethernet, Firewire, modem or digital subscriber line connection. In some embodiments, the communication component 126 can include at least one of a serial port, a parallel port or a USB port.

The memory 124 can include RAM, ROM, one or more hard drives, one or more flash drives or some other suitable data storage elements such as disk drives, etc. The memory 124 may include one or more databases (not shown) for storing information relating to, for example, the patients using the light therapy apparatus 110, different light therapy treatment parameters, and other data related to the operation of the light therapy apparatus 110.

The computing device 140 may be any networked device operable to connect to the network 130. A networked device is a device capable of communicating with other devices through a network, such as the network 130. A network device may couple to the network 130 through a wired or wireless connection.

The computing device 140 may include at least a processor and a memory, and may be an electronic tablet device, a personal computer, workstation, server, portable computer, mobile device, personal digital assistant, laptop, smart phone, WAP phone, an interactive television, video display terminals, gaming consoles, and portable electronic devices or any combination of these.

The computing device 140 can be associated with the light therapy apparatus 110 to enable communication between the computing device 140 and the light therapy apparatus 110. For example, the computing device 140 can be paired to the light therapy apparatus 110 via a local network or via a wireless connection, such as a Bluetooth™ connection.

The network 130 may be any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these, capable of interfacing with, and enabling communication between, the therapy control server 120, the light therapy apparatus 110 and the computing device 140.

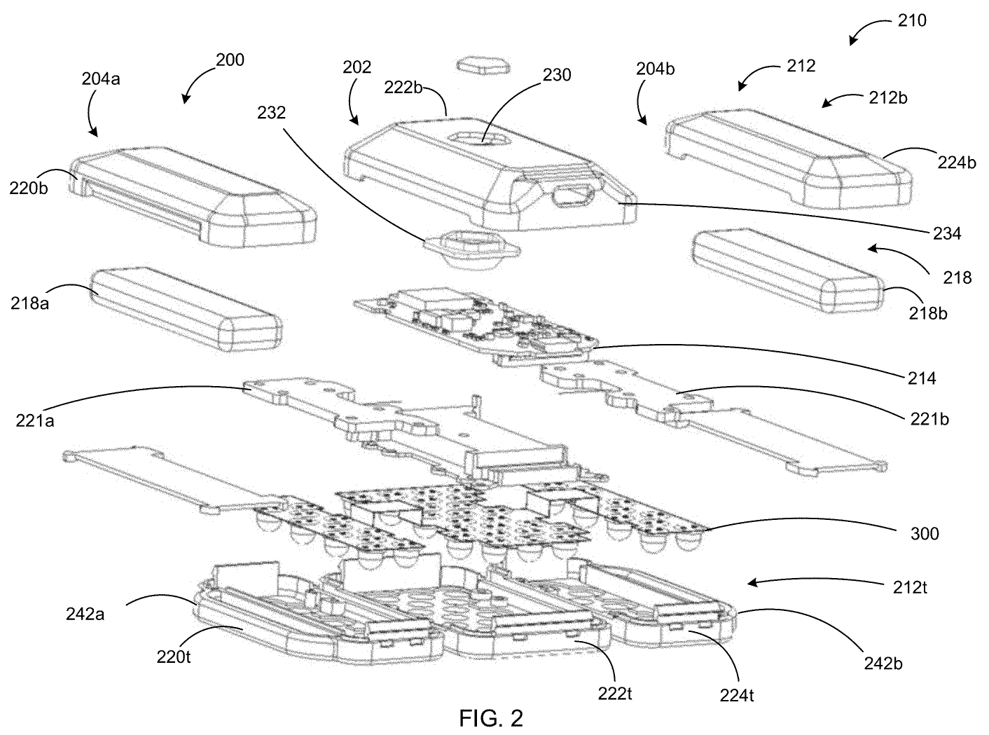

Example embodiments of the light therapy apparatus 110 are shown and will now be described with reference to FIGS. 2 to 12C.

FIG. 2 shows an exploded view 200 of some components of an example light therapy apparatus 210.

The light therapy apparatus 210 includes an enclosure 212 formed of a top portion 212t and a bottom portion 212b. The enclosure 212 can be formed of a relatively rigid material, such as photopolymer resin, polycarbonate, nylon, acrylonitrile butadiene styrene (ABS) plastic, or other similar materials, to protect the electrical components, such as a controller 214 and a circuit board 300. The light therapy apparatus 210 also includes a power supply 218, and flexible members 221a and 221b.

As generally shown in FIG. 2, the light therapy apparatus 210 includes a main chamber 202, a first side chamber 204a that is flexibly mounted to the main chamber 202, and a second side chamber 204b that is flexibly mounted to the main chamber 202. The main chamber 202 is formed with a top main portion 222t and a bottom main portion 222b, the first side chamber 204a is formed with a top first side portion 220t and a bottom first side portion 220b, and the second side chamber 204b is formed with a top second side portion 224t and a bottom second side portion 224b. In the embodiment shown in FIG. 2, the first side chamber 204a is mounted to a first edge of the main chamber 202 and the second side chamber 204b is mounted to the opposite edge of the main chamber 202 (i.e., the second edge of the main chamber).

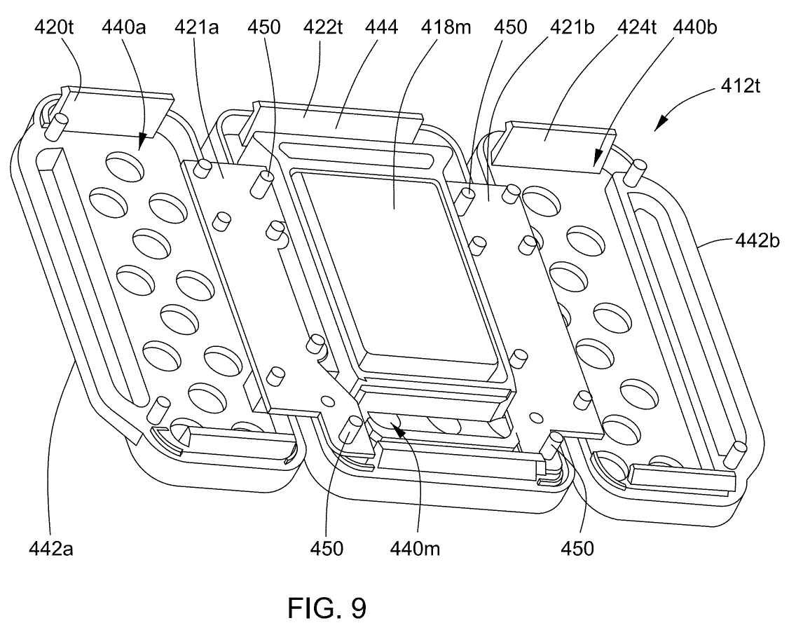

The main chamber 202 in this illustrated embodiment encloses the controller 214, while the first side chamber 204a encloses a first power supply 218a and the second side chamber 204b encloses a second power supply 218b. By including only power supplies in the side chambers, the light therapy apparatus 210 can have a lower and more balanced overall weight. In some embodiments, fewer or more power supplies may be included in the light therapy apparatus 210. For example, in some embodiments, the main chamber 202 can include a main power supply. Components of another example light therapy apparatus are shown in FIG. 9 and shown therein is an example main power supply 418m.

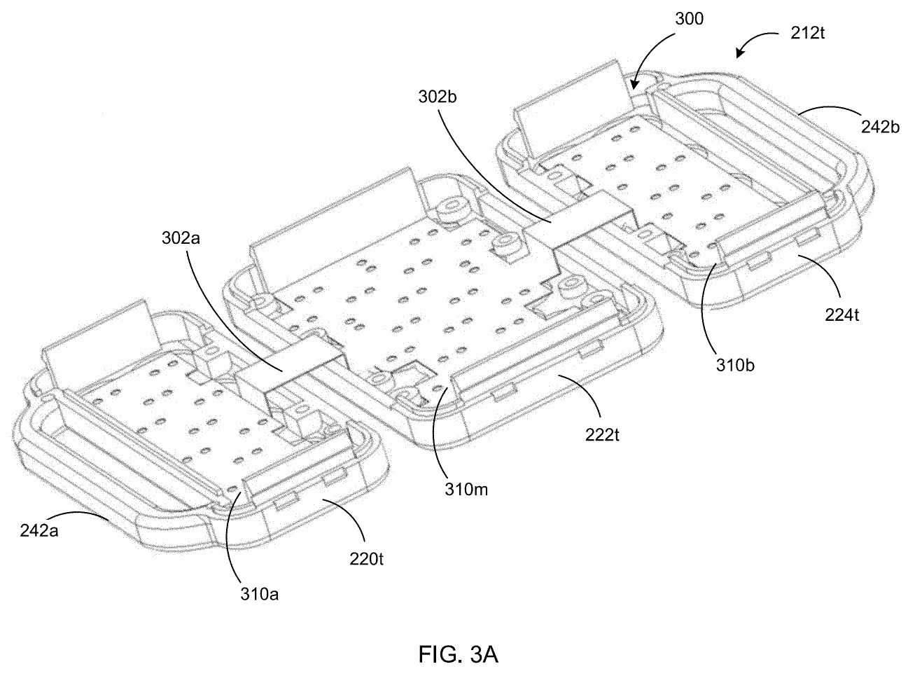

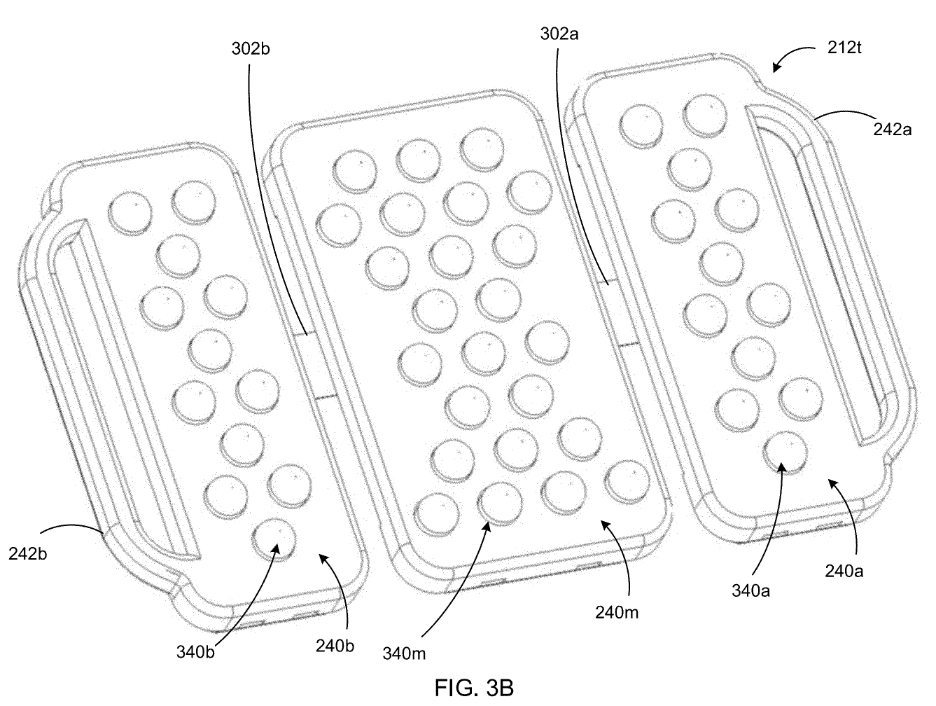

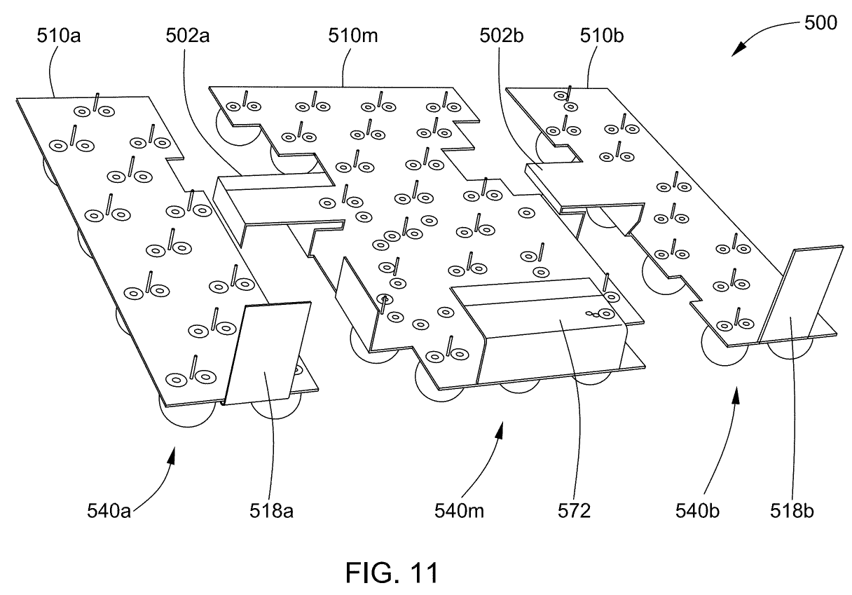

FIGS. 3A and 3B show respective perspective views of the top portion 212t. In FIG. 3A, an inner surface of the top portion 212t is shown coupled with an example circuit board 300. Another example embodiment of the circuit board 300 will be described with reference to FIG. 11. FIG. 3B shows an outer surface of the top portion 212t with arrays of LEDs 340a, 340m and 340b protruding through the respective arrays of openings 240a, 240m, and 240b in the top portion 212t.

The circuit board 300 can be mounted to the top portion 212t of the enclosure 212 by aligning the arrays of LEDs 340a, 340m and 340b with the respective arrays of openings 240a, 240m and 240b so that the arrays of LEDs 340a, 340m and 340b protrude through the respective arrays of openings 240a, 240m and 240b.

The circuit board 300 can be enclosed within the main chamber 202, the first side chamber 204a and the second side chamber 204b. As shown in FIG. 3A, the circuit board 300 includes a main board portion 310m, a first board portion 310a and a second board portion 310b. The circuit board 300 in this example is a flexible printed circuit board. In some embodiments, the circuit board 300 can be provided with a rigid-flex printed circuit board.

The main board portion 310m is partially enclosed within the main chamber 202. One surface of the main board portion 310m is mounted with the main array of light-emitting diodes (LEDs) 340m. The main board portion 310m can also include a controller mating member for receiving a circuit board connector to engage with the controller 214.

The first board portion 310a is partially enclosed within the first side chamber 204a. One surface of the first board portion 310a is mounted with a first side array of LEDs 340a. The first board portion 310a can also include a first power mating member to engage with the first power supply 218a.

The second board portion 310b is similar to the first board portion 310a. The second board portion 310b is partially enclosed within the second side chamber 204b. One surface of the second board portion 310b is mounted with a second side array of LEDs 340b. The second board portion 310b can also include a second power mating member to engage with the second power supply 218b.

The circuit board 300 also includes a first linking member 302a that connects the first board portion 310a to the main board portion 310m, and a second linking member 302b that connects the second board portion 310b to the main board portion 310m.

An external interface opening 234 is also shown in the bottom main portion 222b to allow interfacing between the light therapy apparatus and external devices. For example, the external interface opening 234 can provide access to a Universal Serial Bus (USB) interface. The light therapy apparatus in this embodiment also includes a control button 230 and a status indicator 232. The control button 430 can be a physical push button that, when operated, turns the light therapy apparatus 210 on or off. The control button 230 can also be operated to associate the light therapy apparatus with the corresponding computing device 140.

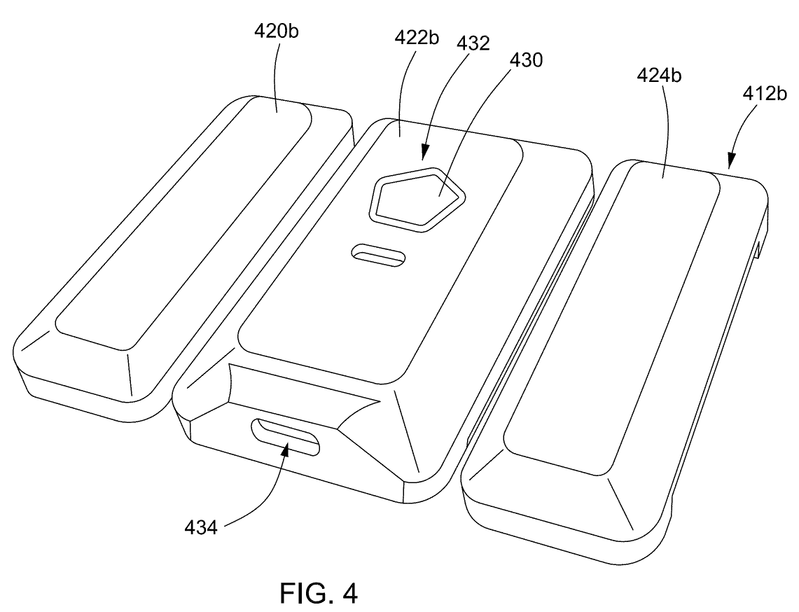

Another example light therapy apparatus will now be described with reference to FIGS. 4 to 9.

FIG. 4 is a rear perspective view of an example bottom portion 412b. As shown in FIG. 4, similar to the light therapy apparatus 210 shown in FIG. 2, an external interface opening 434 is formed in the bottom main portion 422b to allow interfacing between the light therapy apparatus and external devices. For example, the external interface opening 434 can provide access to a Universal Serial Bus (USB) interface. The light therapy apparatus in this embodiment also includes a control button 430 and a status indicator 432. Like the control button 230, the control button 430 can be a physical push button that, when operated, turns the light therapy apparatus on or off. The control button 430 can also be operated to associate the light therapy apparatus with the corresponding computing device 140.

In the example shown in FIGS. 2 and 4, the status indicator 232, 432 is shown as a pentagon but it will be understood that the example status indicator 232, 432 is only shown for illustrative purposes and is not intended as a limitation. Different configuration of the status indicator 232, 432 can be used. The status indicator 232, 432 can be operated by the controller 214 to display a different colour depending on a power supply level and/or an operation status. Example operation statuses can include a pairing status, a standby status, a powered down status, a low power status, a charging status and an in-treatment status. For example, when the light therapy apparatus lacks sufficient power to complete a light therapy treatment, the status indicator 432 can flash a designated colour, such as red. In alternative embodiments, the status indicator 432 can flash at different intervals to represent different statuses.

In some embodiments, the status indicator 432 is formed with a light guide. A portion of the status indicator 432 can be translucent, to a degree, to allow light to pass from inside the light therapy apparatus outwards. For example, the status indicator 432 can diffuse light from a light emitting diode (LED) (e.g., a RGB LED) located inside the light therapy apparatus.

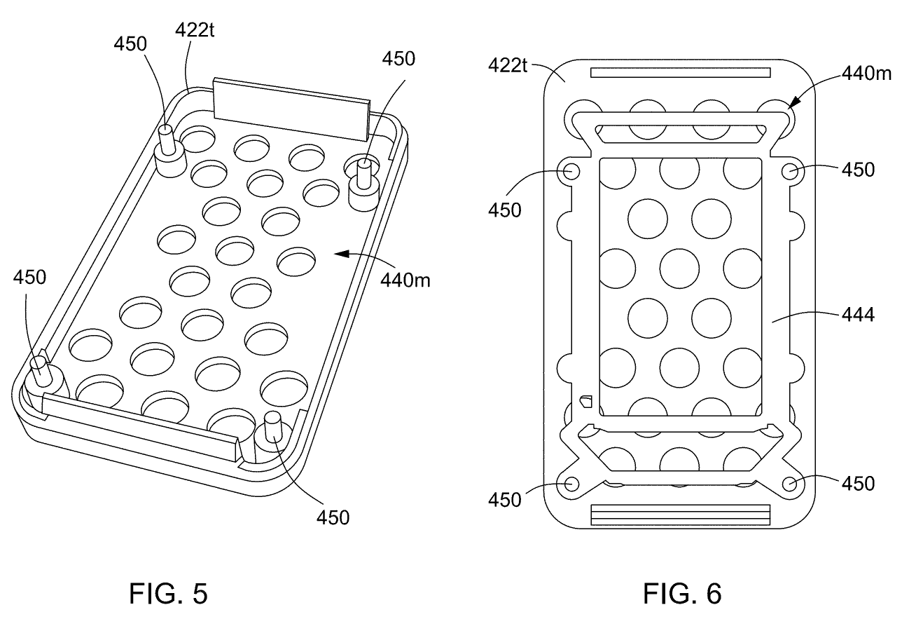

FIG. 5 is a rear perspective view of an inner surface of the top main portion 422t. The top main portion 422t has an array of openings 440m through which a respective array of LEDs protrude. As will be described with reference to FIG. 11, the respective array of LEDs can be coupled to the circuit board.

In the example shown in FIG. 5, the array of openings 440m has a pattern analogous to an hour-glass. The hour-glass pattern is pinched near the center of the top main portion 422t to accommodate the linking members of the circuit board at the interface between the main chamber and the first side chamber, and the main chamber and the second side chamber. The hour-glass pattern of the array of openings 440m is merely an example and is not a limitation to the arrangement of the array of openings 440m. Other patterns of the array of openings 440m that can accommodate the linking members of the circuit board can similarly be used.

The top main portion 422t also includes posts 450 for coupling the top main portion 422t to the top first side portion 420t and the top second side portion 424t. Briefly, as shown in FIG. 9, the top first side portion 420t and the top second side portion 424t are coupled to the top main portion 422t with respective flexible members 421a and 421b with, at least, the posts 450.

The posts 450 can also help to minimize torsion at the main and side chambers when the light therapy apparatus is being used. In some cases, the light therapy apparatus may be strapped tightly around the body of the patient, which can cause torsion in the main and side chambers if not properly supported.

FIG. 6 shows the top main portion 422t of FIG. 5 coupled with an optional support member 444. As shown in FIG. 6, the support member 444 is mounted to the top main portion 422t with at least the posts 450. When the light therapy apparatus is assembled, the support member 444 can apply pressure against the circuit board 300 with the arrays of LEDs mounted thereon. When the arrays of LEDs are coupled to the top portion 412t, the arrays of LEDs protrude through the respective arrays of openings 440a, 440m and 440b. It is, therefore, possible for an external force to be applied to the protruded arrays of LEDs and cause movement in the protruded arrays of LEDs. The support member 444 can, therefore, help to resist any movement of the arrays of LEDs that may be caused by the external force.

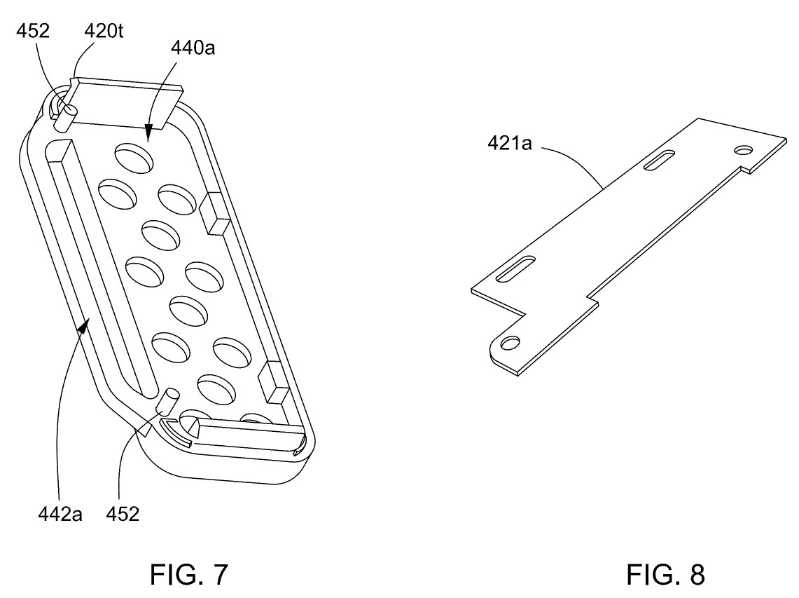

FIG. 7 is a rear perspective view of an inner surface of the top first side portion 420t. The top first side portion 420t has an array of openings 440a through which a respective array of LEDs protrude. FIG. 7 also shows posts 452, which can facilitate alignment of the components of the light therapy apparatus. In some embodiments, the posts 452 can instead be openings with pins inserted therein. In some embodiments, no posts 452 or openings with pins can be provided.

The top first side portion 420t also has an anchor member 442a through which one side of a strap can be attached. The light therapy apparatus can be worn with the strap. The strap can be made with washable materials. The strap can also be secured around the body with a fastener, such as Velcro™, buttons, etc.

As shown in FIG. 9, the top second side portion 24t is similar to the top first side portion 420t shown in FIG. 7. The top second side portion 424t also includes an array of openings 440b through which a respective array of LEDs protrude. An anchor member 442b is also provided at the top second side portion 424t to receive the other side of the strap.

FIG. 8 is a perspective view of the flexible member 421a. Flexible member 221a shown in FIG. 2 is similar to flexible member 421a. As shown in FIG. 9, flexible members 421a and 421b couple the top first side portion 420t to the top main portion 422t, and the top second side portion 424t to the top main portion 422t, respectively. The flexible members 421a and 421b can allow the enclosure portions 420t, 422t and 424t to be flexibly mounted to each other. During a light therapy treatment, for example, the flexible members 421a and 421b can allow the light therapy apparatus to fit to the body of the patient.

The flexible members 421a and 421b can also at least partially shield the linking members of the circuit board. By shielding the linking members, the flexible members 421a and 421b can protect the linking members from being damaged. The flexible members 421a and 421b can also minimize the mechanical load that may be applied at the interface between the main and side chambers, as well as any mechanical load that may be applied to the circuit board during the light therapy treatment.

In some embodiments, the flexible members 421a and 421b can be formed of abrasion resistant polyurethane sheets, neoprene, thermoplastic elastomer (TPE), or other similar materials.

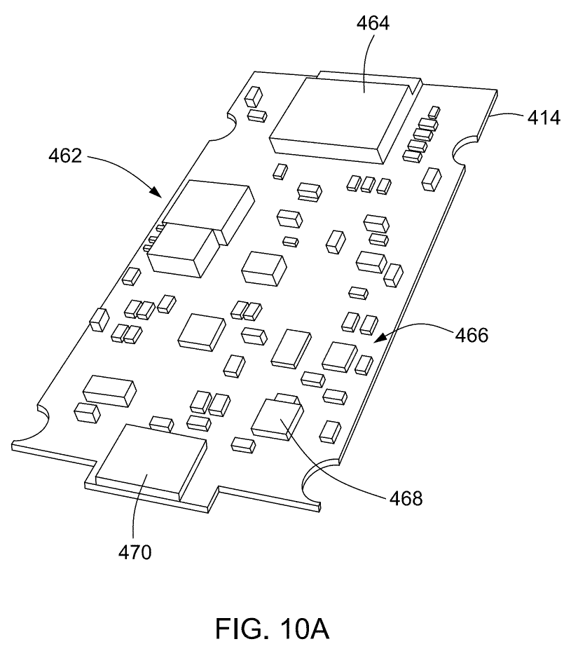

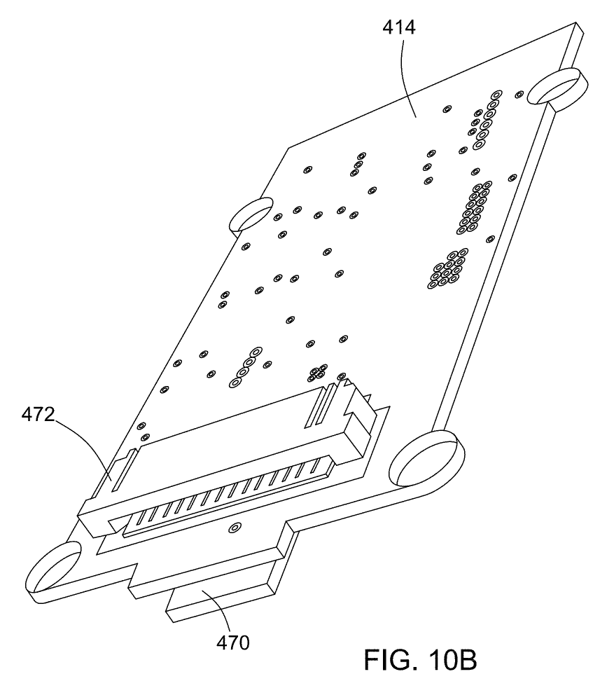

Reference will now be made to FIGS. 10A and 10B.

FIG. 10A is a perspective front view of an example controller 414 and FIG. 10B is a perspective rear view of the controller 414. The controller 414 of FIGS. 10A and 10B is an illustrative example and is not intended to limit the configuration and/or implementation of the controller 414. In this example embodiment the controller 414 is provided with a rigid printed circuit board (PCB).

As shown in FIG. 10A, the controller 414 includes a LED driving component 462, a communication controller 464 (e.g., a Bluetooth controller), a power supply monitoring component 466, a power supply charging component 468, and a USB interface component 470.

The LED driving component 462 can control the operation of the one or more arrays of LEDs within the light therapy apparatus. The LED driving component 462 can regulate the power delivered to each array of LEDs. For example, by regulating the power delivered to each array of LEDs, the LED driving component 462 can vary one or more parameters of the array of LEDs, and thus, the overall light therapy treatment, such as frequency, duty cycle, and intensity. The frequency, duty cycle and/or intensity of each array of LEDs can vary with the type of injury that the light therapy apparatus is treating.

In some embodiments, the LED driving component 462 can include one or more channels with each channel operating a respective array of LEDs. Each channel of the LED driving component 462 can operate the respective array of LEDs differently than another channel, for example.

In the embodiment shown in FIGS. 10A and 10B, it can be seen that the LED driving component 462 is positioned relatively close to a circuit board connector 472, which interfaces with the circuit board. By positioning the LED driving component 462 relatively close to the circuit board connector 472, the copper trace length can be reduced. The LED driving component 462 is also positioned in proximity to the true ground of the circuit board, which can, to an extent, minimize signal noise.

The Bluetooth controller 464 can be positioned away from the USB interface component 470 to minimize any signal interference resulting from the high power at the USB interface component 470. By positioning the Bluetooth controller 464 away from the USB interface component 470, interference between the Bluetooth antenna and the copper traces may be minimized.

The power supply monitoring component 466 and the power supply charging component 468 can also be positioned in proximity to the circuit board connector 472 as the power supplies 418 will couple to the circuit board.

The USB interface component 470 provides a USB interface for the light therapy apparatus.

In some embodiments, the controller 414 can include a motion sensor for detecting a user movement, such as a range of motion of a joint. The motion sensor can include, but is not limited to, an accelerometer, tilt sensor, force sensor, pressure sensor, and/or a gyroscope.

Continuing now to FIG. 11, which shows an example circuit board 500 for the light therapy apparatus. Similar to the circuit board 300, the circuit board 500 can be enclosed within the main chamber, the first side chamber and the second side chamber.

The circuit board 500 includes a main board portion 510m, a first board portion 510a and a second board portion 510b. The circuit board 500 in this example is a flexible printed circuit board. In some embodiments, the circuit board 500 can be provided with a rigid-flex printed circuit board.

The main board portion 510m is partially enclosed within the main chamber. As shown in FIG. 11, one surface of the main board portion 510m is mounted with a main array of light-emitting diodes (LEDs) 540m and another surface of the main board portion 510m has a controller mating member 572 for receiving the circuit board connector to engage with the controller 214, 414.

The first board portion 510a is partially enclosed within the first side chamber. One surface of the first board portion 510a is mounted with a first side array of LEDs 540a and another surface of the first board portion 510a has a first power mating member 518a to engage with the first power supply.

The second board portion 510b is similar to the first board portion 510a. The second board portion 510b is partially enclosed within the second side chamber. One surface of the second board portion 510b is mounted with a second side array of LEDs 540b and a second power mating member 518b to engage with the second power supply.

In some embodiments, each of the arrays of LEDs 540a, 540m and 540b can include one or more LEDs with an operating wavelength in a range of approximately 800 nm to 940 nm. This range of operating wavelength has been shown to be effective in healing medical conditions related to the musculoskeletal system.

In some embodiments, each LED in the arrays of LEDs 540a, 540m and 540b can emit approximately 22 mW of radiant power. The amount of radiant power emitted by each LED in the arrays of LEDs 540a, 540m and 540b can be adjusted by the therapy control server 120.

In some embodiments, the density of the number of LEDs in each of the arrays of LEDs 540a, 540m and 540b is approximately two LEDs for each surface area of 1 cm².

The circuit board 500 also includes a first linking member 502a that connects the first board portion 510a to the main board portion 510m, and a second linking member 502b that connects the second board portion 510b to the main board portion 510m.

With reference now to FIGS. 9 and 11, the circuit board 500 can be mounted to the top portion 412t of the enclosure 412 by aligning the arrays of LEDs 540a, 540m and 540b with the respective arrays of openings 440a, 440m and 440b so that the arrays of LEDs 540a, 540m and 540b protrude through the respective arrays of openings 440a, 440m and 440b. When the circuit board 500 is mounted to the top portion 212t, the flexible members 421a and 421b can shield the respective first linking member 502a and second linking member 502b.

In some embodiments, a shielding layer can be mounted to the top portion 412t of the enclosure 412 prior to the circuit board 500. Similar to the bottom portion 412b, the shielding layer includes arrays of openings through which the arrays of LEDs 540a, 540m and 540b can protrude. The shielding layer can be partially enclosed within the main chamber, the first side chamber and the second side chamber. An example shielding layer can include a linking member shield portion that at least partially shields the first linking member 502a and the second linking member 502b. The shielding layer, therefore, can provide additional protection to the first linking member 502a and the second linking member 502b.

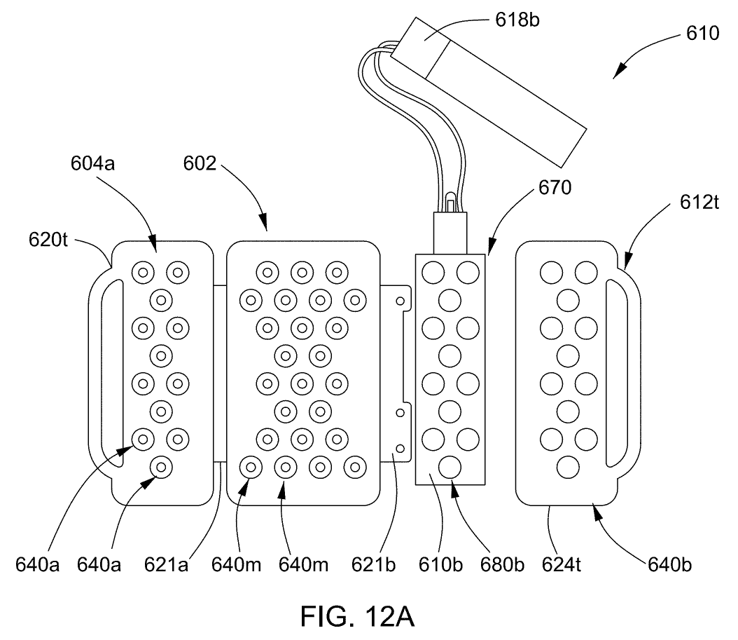

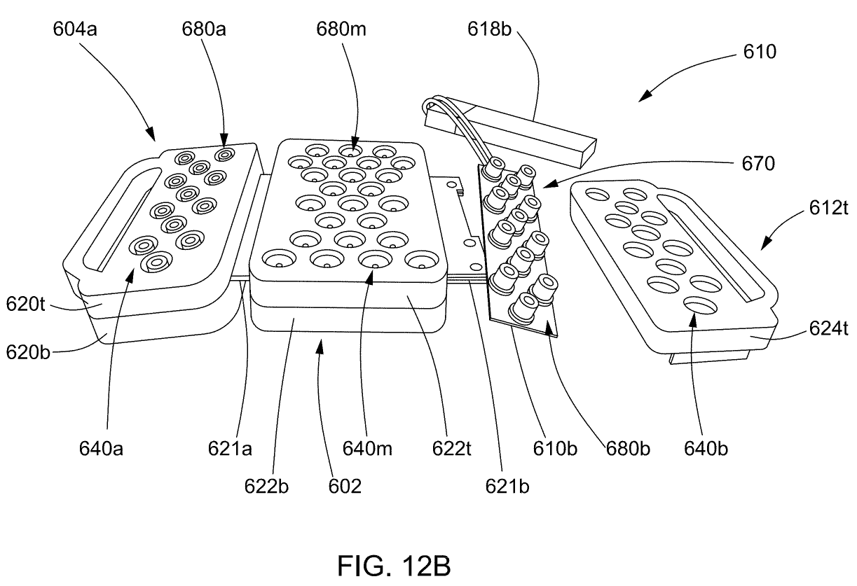

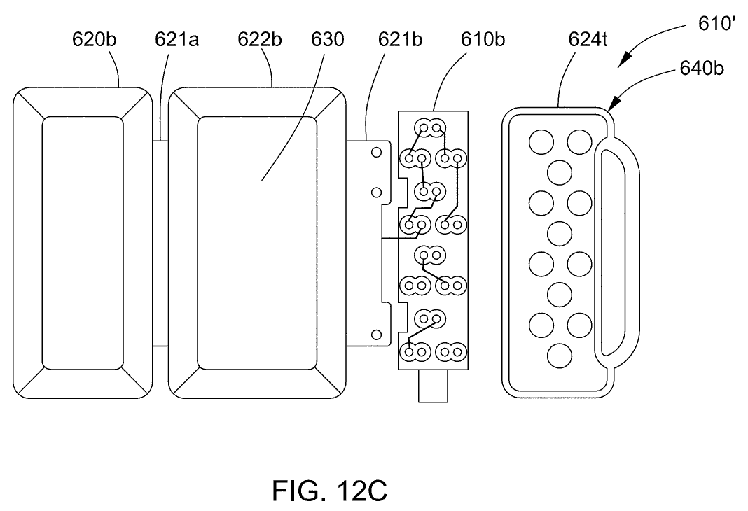

Another embodiment of the light therapy apparatus 610 will now be described with reference to FIGS. 12A to 12C.

FIG. 12A is a front view of the light therapy apparatus 610 with some components of a second side chamber exposed. FIG. 12B is a side perspective view of the light therapy apparatus 610 of FIG. 12A and FIG. 12C is a partial rear view 610′ of the light therapy apparatus 610 of FIG. 12A.

Similar to the light therapy apparatus 210, the light therapy apparatus 610 includes a first side chamber 604a, a main chamber 602 and a second side chamber. Each of the first side chamber 604a, the main chamber 602 and the second side chamber is formed with a top portion and a bottom portion. The first side chamber 604a is formed by mounting together portions 620t and 620b, the main chamber 602 is formed by mounting together portions 622t and 622b, and the second side chamber 604b is formed by mounting together portions 624t and a bottom portion not shown.

Arrays of LEDs 680a and 680m protrude through the top portion 612t of the light therapy apparatus 610 via respective arrays of openings 640a and 640m. For illustrative purposes, the array of LEDs 680b, which is mounted to a second board portion 610b of a circuit board 670, is shown separate from the second top side portion 624t. Each of the first side chamber 604a and the second side chamber 604b includes a power supply (only power supply 618b is shown in FIGS. 12A and 12B as an example).

The example light therapy apparatus 610 includes forty-eight LEDs with twelve LEDs at each of the first side chamber 604a and the second side chamber 604b, and twenty-four LEDs at the main chamber 602. The protruded LEDs can make direct contact with the body of the user, which can minimize light energy losses.

In FIG. 12C, it is shown that the light therapy apparatus 610 includes a control button 630 similar to control button 430. A rear surface of the second board portion 610b is also shown.

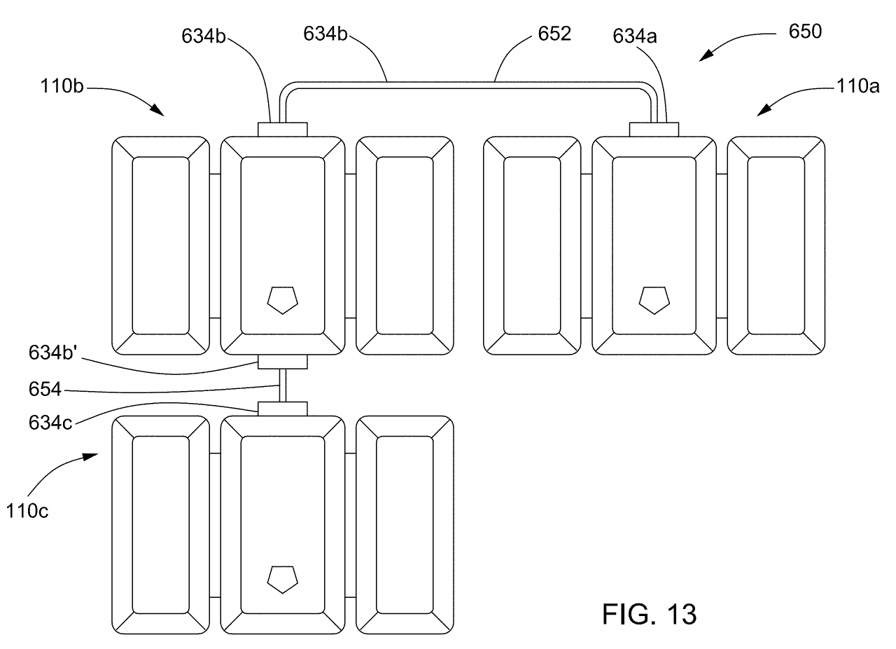

Reference will now be made to FIG. 13, which shows an example chain 650 of light therapy apparatuses 110a, 110b, 110c. The illustrated chain 650 is only an example. The chain 650 can include a fewer number or a greater number of light therapy apparatuses 110, as appropriate for the surface receiving the light therapy treatment. Although shown slightly different in FIG. 13, each of the illustrated light therapy apparatuses 110a, 110b, 110c can be analogous in structure and functionality to each other.

To form the chain 650, the light therapy apparatuses 110a, 110b, 110c can be connected to each other via connection cables at respective external interface openings. As shown in FIG. 13, the light therapy apparatus 110a can be connected to the light therapy apparatus 110b with a connection cable 652 between respective external interface openings 634a and 634b. To accommodate more than one connection, a light therapy apparatus, such as 110b, can include multiple external interface openings, such as 634b and 634b′. The light therapy apparatus 110b connects to the light therapy apparatus 110c with a connection cable 654 between respective external interface openings 634b′ and 634c.

The connection cables 652, 654 can be coupled to the external interface opening with a magnetic coupling, in some embodiments. Other types of connections can be used.

The arrays of LEDs 340a, 340m, 340b at the light therapy apparatuses 110a, 110b, 110c can be centrally controlled by one or more of the light therapy apparatuses 110a, 110b, 110c. For example, in the example shown in FIG. 13, the light therapy apparatus 110b can act as a central apparatus in the chain 650 and transmit the operating signals for the arrays of LEDs 340a, 340m, 340b at the light therapy apparatuses 110a, 110c. In other embodiments, the light therapy apparatus 110a or the light therapy apparatus 110c can act as the central apparatus.

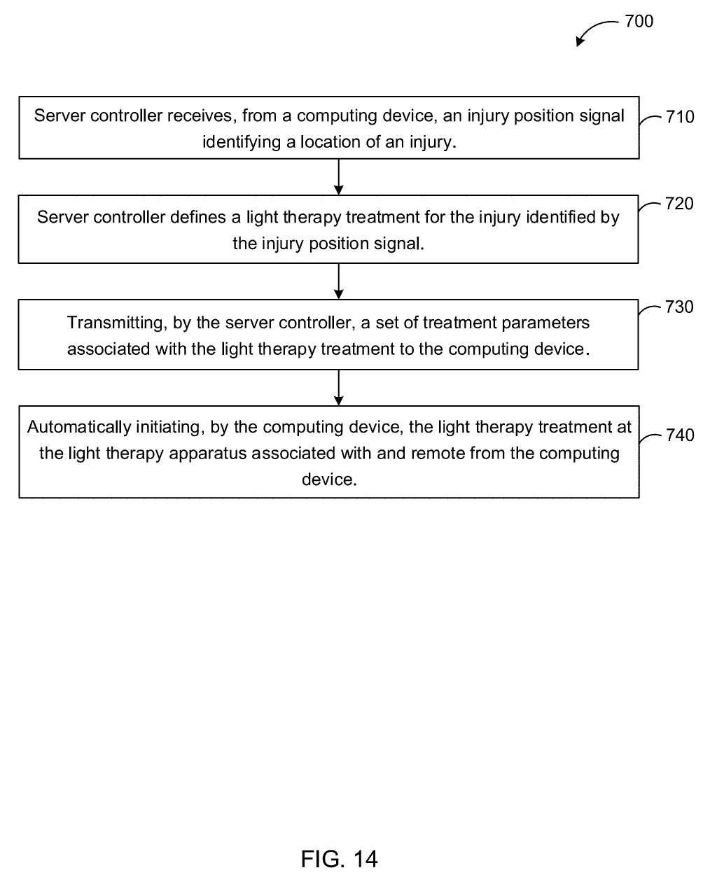

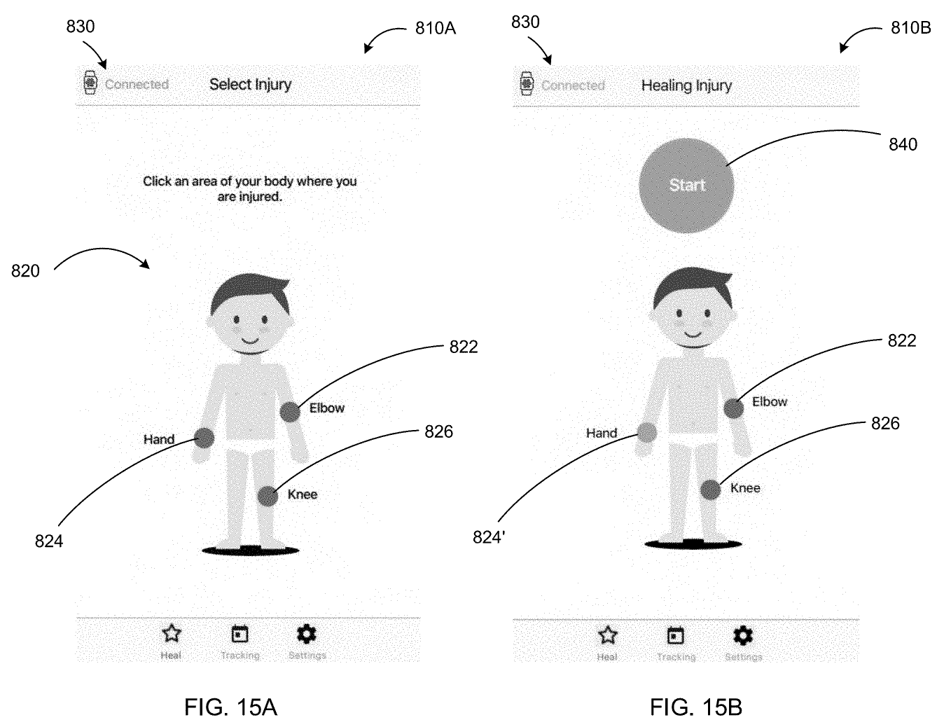

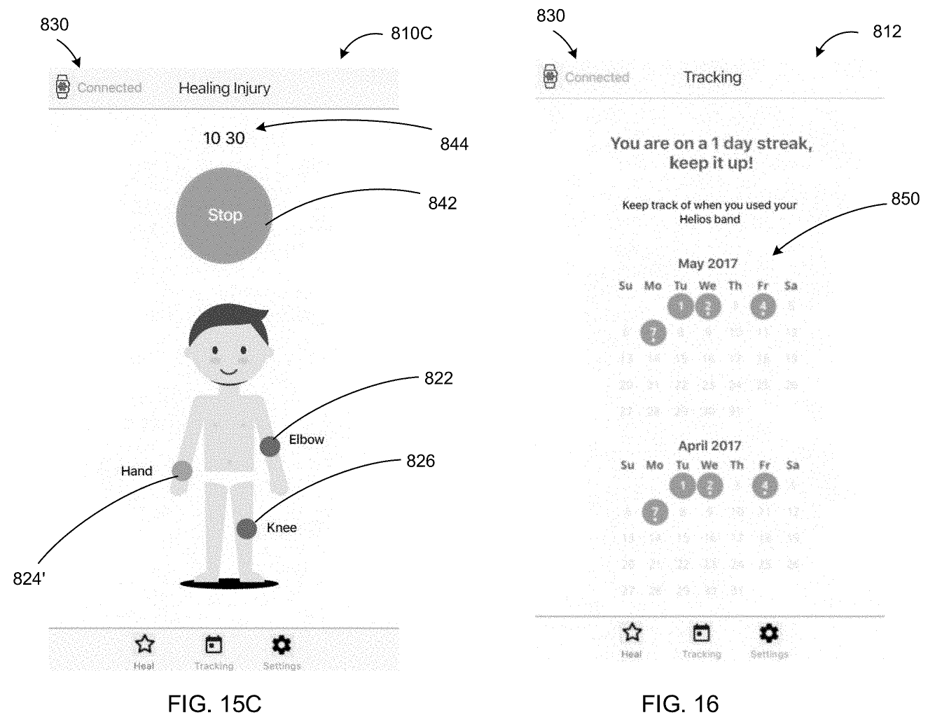

FIG. 14 is a flowchart of an example method 700 of operating the light therapy apparatus, such as 110, 210 and 610, disclosed herein. To illustrate the method 700, reference will be made simultaneously to FIGS. 15A to 15C and 16 and to the example light therapy apparatuses 110, 210. It will be understood that the light therapy apparatus 610 can similarly be operated with method 700.

At 710, the server controller 122 receives, from the computing device 140, an injury position signal identifying a location of an injury.

FIG. 15A is a screenshot of an example injury identification interface 810A. As shown in FIG. 15A, a connected status icon 830 shows that the computing device 140 is associated with the light therapy apparatus 110.

The injury identification interface 810A can illustrate a patient representative image 820 (e.g., a human) with location indicators 822, 824 and 826 that can be selected by the user to identify the location of the injury. Although only location indicators 822, 824 and 826 are shown in the injury identification interface 810A, fewer or more location indicators can be shown in some embodiments. In some embodiments, the patient representative image 820 may not show any location indicators and the user can select the location without the assistance of any location indicators. When one or more of the location indicators 822, 824 and 826 is selected, the computing device 140 generates the injury position signal identifying the location corresponding to the selected location indicator and transmits the injury position signal to the therapy control server 120.

In some embodiments, the injury position signal can include a user identifier corresponding to a user with the injury, or the patient to receive light therapy treatment from the light therapy apparatus 110. It is possible that the patient receiving the light therapy treatment is different from the user operating the computing device 140. For example, an elderly patient may be assisted by another.

At 720, the server controller 122 defines a light therapy treatment for the injury identified by the injury position signal.

The light therapy treatment defines various parameters, such as an intensity, a duration and/or a wavelength, for the arrays of LEDs 540a, 540m and 540b at the light therapy apparatus 110. The values of the parameters can vary with different factors, such as the location of the injury, the type of user (e.g., age of the user), the length of the injury, etc.

In some embodiments, the server controller 122 can define the light therapy treatment by retrieving a user profile stored in the memory 124 remote from the computing device 140. From the retrieved user profile, the server controller 122 can identify any similar injuries suffered by that user. The server controller 122 can then define the light therapy treatment based at least on a treatment history associated with the user profile.

For example, the server controller 122 can determine from the user profile that the user suffered a prior injury that is similar to the currently identified injury. The server controller 122 can review the recovery progress of the prior injury when defining the light therapy treatment for the current injury. For example, the server controller 122 can consider the length of treatment required for treating the prior injury and if the prior injury was treated within a treatment length threshold, the server controller 122 can define the light therapy treatment for the current injury to include the same parameters as the treatment used for the prior injury. However, if the prior injury was not treated within the treatment length threshold, the server controller 122 can define different parameters for the light therapy treatment to be applied to the current injury. For example, the server controller 122 can define the light therapy treatment for the current injury to include treatment parameters with increased values relative to the light therapy treatment applied to the previous injury.

The treatment length threshold can include a number of days, in some embodiments.

In some embodiments, the server controller 122 can define the light therapy treatment by determining whether an ongoing treatment is available for the injury. If the server controller 122 determines an ongoing treatment is available, the server controller 122 determines whether to continue the ongoing treatment.

For example, the server controller 122 can determine a recovery progress of the injury with the ongoing treatment. To determine the recovery progress, the server controller 122 can generate a progress assessment interface at the computing device 140 requesting an assessment of the recovery progress. The assessment can be user-generated or determined from measurements conducted by the light therapy apparatus 110. From the progress assessment interface, the server controller 122 can receive a rating on various aspects of the injury and recovery, such as a pain level (e.g., on a scale of 1 to 10, etc.), a range of motion, a strength of the injured location, and/or a stiffness of the injured location. Certain aspects of the injury/recovery can be measured through an exercise shown on the computing device 140.

In some embodiments, the light therapy apparatus 110 can include a motion sensor, such as an accelerometer, a gyroscope or other similar devices, for measuring the range of motion at the location of injury.

After receive the rating(s) via the progress assessment interface, the computing device 140 can generate and transmit a recovery assessment signal representing the recovery progress of the injury to the server controller 122. The server controller 122 can then adjust the ongoing treatment for the injury based on the recovery progress of the injury.

At 730, transmit, by the server controller 122, a set of treatment parameters associated with the light therapy treatment to the computing device 140.

FIG. 15B is a screenshot of an example treatment initiation interface 810B.

As shown in FIG. 15B, location indicator 824′ is selected, which indicates that the injured location is at the right hand. After receiving the set of treatment parameters for the injured location from the server controller 122, the computing device 140 can then prompt the user to put the light therapy apparatus 110 over the injured area and secure the light therapy apparatus 110 thereon. When secured, the user can select the start control 840 to initiate the light therapy treatment.

At 740, automatically initiating, by the computing device 140, the light therapy treatment at the light therapy apparatus 110 associated with and remote from the computing device 140.

FIG. 15C is a screenshot of an example treatment termination interface 810C.

While the light therapy treatment is taking place, the treatment termination interface 810C can be available so that the user can choose to terminate the treatment at any time by initiating a stop control 842. During the light therapy treatment, a timer 844 can be displayed to illustrate the amount of time remaining in the light therapy treatment. When the timer 844 completes the countdown, the light therapy apparatus 110 will automatically shut off and a treatment completion notification will be provided to user.

In some embodiments, prior to initiating the light therapy treatment at the light therapy apparatus 110, the server controller 122 can operate the computing device 140 to detect a power supply level at the light therapy apparatus 110. In response to determining the power supply level is below a minimum power threshold, the server controller 122 can transmit a power deficient notification to the computing device 140, which can prevent the computing device 140 from automatically initiating the light therapy treatment at the light therapy apparatus 110. When the power deficient notification is generated, the computing device 140 and/or the server controller 122 can trigger the user to charge the light therapy apparatus 110 before allowing for the light therapy treatment to take place.

FIG. 16 is a screenshot of an example treatment progress interface 812.

The treatment progress interface 812 can illustrate to the patient a frequency of usage of the light therapy apparatus 110. For example, a calendar display 850 is shown in the treatment progress interface 812 with the days circled as the days on which the patient completed a light therapy treatment with the light therapy apparatus 110. Other displays may be provided to illustrate the frequency of usage.

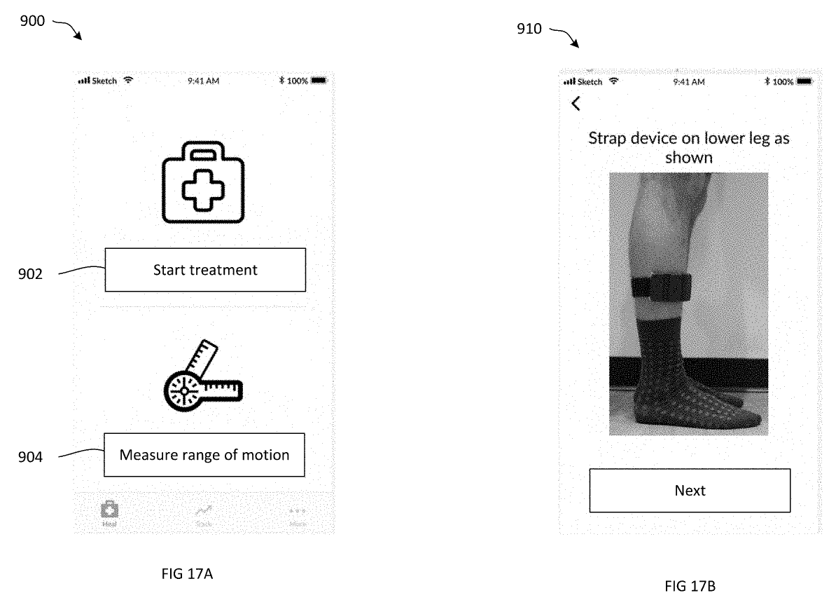

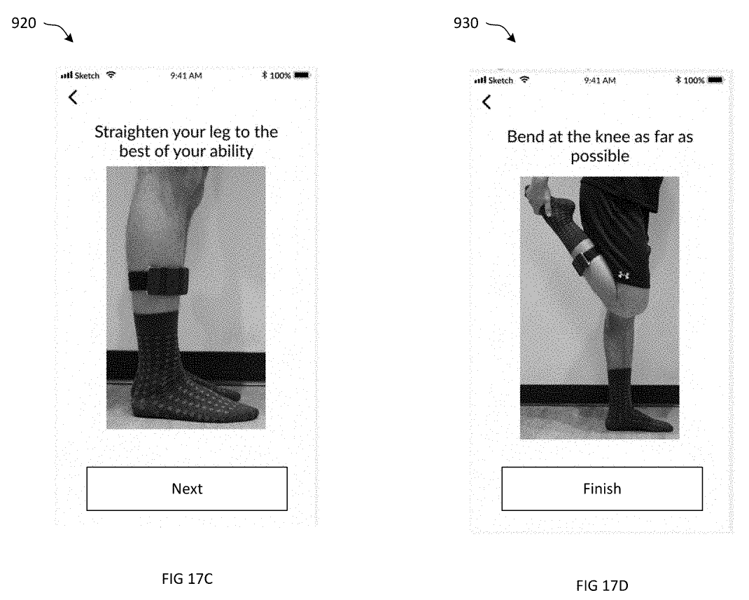

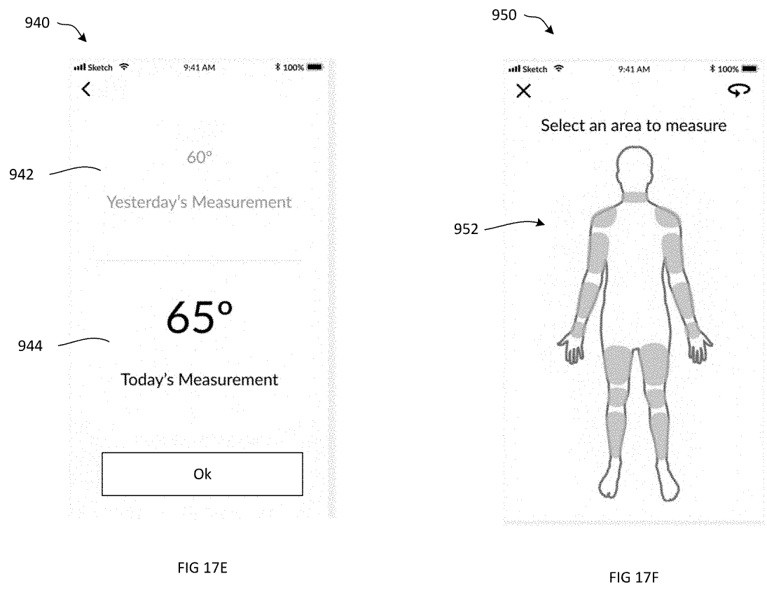

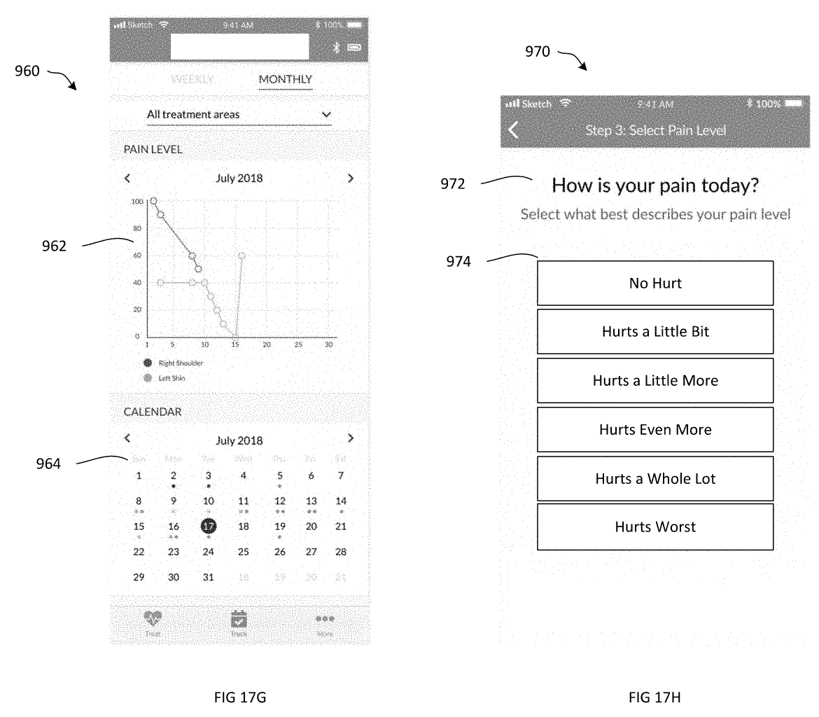

FIGS. 17A to 17H are screenshots of example treatment progress interfaces 900, 910, 920, 930, 940, 950, 960, 970.

At 900, the screen displays an option to start treatment 902 or measure range of motion 904. Where range of motion 904 is selected, the user is provided a display 910 to straps the device onto their body part, at. The user is provided a display 920 to straighten the joint and a display 930 to move the joint through the range of motion. The system calculates the range of motion and the screen displays the range of motion measurement 944 and may also display a previous measurement 942. The user may be provided 950 with a selection of a particular body part to measure the range of motion of with a display of the body 952.

The screen may also display a periodic (e.g., weekly, monthly) report 960 on the performance of a user metric (e.g., range of motion, pain) 962. The performance of the user metric may be selected for different days in a calendar 964.

The screen may also display, at 970, a question 972 to the user on their treatment with a plurality of feedback selections to respond to 974. This feedback 974 may be used to monitor the treatment, and inform an adjustment of the treatment regimen.

In some embodiments, the therapy control server 120 can monitor the frequency of usage of the light therapy apparatus 110 by the user. When the therapy control server 120 detects a consistent usage for a period of time and a sudden termination of usage, the therapy control server 120 can trigger a treatment reminder at the computing device 140. The consistent usage can vary for a user. For example, to determine a pattern of usage for the user, the therapy control server 120 can monitor usage of the light therapy apparatus 110 by the user for a usage period, such as three to six months (or other periods of time). From monitoring the usage during that usage period, the therapy control server 120 can determine whether the user's usage of the light therapy apparatus 110 has a pattern, such as daily, every two days, on weekends, weekly, etc. If any pattern is detected, the therapy control server 120 can track the user's future usage based on that pattern and generate the treatment reminder when the user departs from that pattern.

In some embodiments, the therapy control server 120 may not generate any treatment reminders for a period of time after detecting a lack of usage for a period. After a further period of no usage, the therapy control server 120 can generate a treatment reminder.

In some embodiments, the therapy control server 120 can provide other user interfaces via the computing device 140 for providing information and/or advertisements. The information and/or advertisements provided can depend on the user profiles and/or the injuries experienced by the user.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements. Furthermore, the term “body” typically refers to the body of a patient, a subject or an individual who receives treatment from the light therapy apparatus. The patient or subject is generally a human or other animal.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the drawings, the illustrated user interface text and controls are provided as examples only and are not meant to be limiting. Other suitable user interface elements may be possible.

We claim:

1. A light therapy apparatus comprising:
a main chamber comprising a controller;
a first side chamber flexibly mounted to the main chamber, the first side chamber comprising a first power supply;
a second side chamber flexibly mounted to the main chamber, the second side chamber comprising a second power supply; and
a circuit board partially enclosed within the main chamber, the first side chamber and the second side chamber, the circuit board comprising:
a main board portion partially enclosed within the main chamber, the main board portion comprising a main array of light-emitting diodes (LEDs) and a controller mating member to engage with the controller;
a first board portion partially enclosed within the first side chamber, the first board portion comprising a first side array of LEDs and a first power mating member to engage with the first power supply;
a second board portion partially enclosed within the second side chamber, the second board portion comprising a second side array of LEDs and a second power mating member to engage with the second power supply;
a first linking member connecting the first board portion to the main board portion; and
a second linking member connecting the second board portion to the main controller portion.

2. The light therapy system of claim 1, wherein:
each of the main board portion, the first board portion and the second board portion has an array of openings corresponding to the respective main array of LEDs, the first side array of LEDs and the second side array of LEDs, and each of the main array of LEDs, the first side array of LEDs and the second side array of LEDs protrude through the respective array of openings.

3. The light therapy system of claim 1, wherein:
the first side chamber is mounted to the main chamber with a first flexible member; and
the second side chamber is mounted to the main chamber with a second flexible member.

4. The light therapy system of claim 3, wherein:
the first flexible member at least partially shields the first linking member; and
the second flexible member at least partially shields the second linking member.

5. The light therapy system of claim 3 further comprising:
a shielding layer partially enclosed within the main chamber, the first side chamber and the second side chamber, wherein the shielding layer at least partially shields the first linking member and the second linking member.

6. The light therapy system of claim 1, wherein the circuit board comprises a flexible printed circuit board.

7. The light therapy system of claim 1, wherein:
the first side chamber is mounted to a first edge of the main chamber and the second side chamber is mounted to a second edge of the main chamber, the second edge being opposite to the first edge.

8. The light therapy system of claim 1, wherein:
the controller comprises a LED driving component to control an operation of each of the main array of LEDs, the first side array of LEDs and the second side array of LEDs.

9. The light therapy system of claim 1, wherein:
each of the main array of LEDs, the first side array of LEDs and the second side array of LEDs comprises one or more LEDs with an operating wavelength in a range of approximately 800 nm to 940 nm.

10. The light therapy system of claim 1, wherein:
each of the main array of LEDs, the first side array of LEDs and the second side array of LEDs comprises approximately two LEDs for each surface area of 1 cm².

11. A light therapy system comprising:
a light therapy apparatus;
a computing device associated with the light therapy apparatus; and
a therapy control server in communication with the light therapy apparatus and the computing device, the therapy control server comprising:
a server controller operable to control a light therapy session at the light therapy apparatus;
a communication component to transmit and receive operation signals to and from the computing device; and
a memory to store operating data in respect of the operation of the light therapy apparatus.

12. The system of claim 11, wherein the light therapy apparatus comprises:
a main chamber comprising a controller;
a first side chamber flexibly mounted to the main chamber, the first side chamber comprising a first power supply;
a second side chamber flexibly mounted to the main chamber, the second side chamber comprising a second power supply; and
a circuit board partially enclosed within the main chamber, the first side chamber and the second side chamber, the circuit board comprising:
a main board portion partially enclosed within the main chamber, the main board portion comprising a main array of light-emitting diodes (LEDs) and a controller mating member to engage with the controller;
a first board portion partially enclosed within the first side chamber, the first board portion comprising a first side array of LEDs and a first power mating member to engage with the first power supply;
a second board portion partially enclosed within the second side chamber, the second board portion comprising a second side array of LEDs and a second power mating member to engage with the second power supply;
a first linking member connecting the first board portion to the main board portion; and
a second linking member connecting the second board portion to the main controller portion.

13. A method of operating a light therapy apparatus, the method comprising:
receiving, from a computing device, an injury position signal identifying a location of an injury;
operating a server controller remote to the computing device to define a light therapy treatment for the injury identified by the injury position signal, the light therapy treatment defining at least one of an intensity, a duration and a wavelength of an array of light emitting diodes (LEDs) at a light therapy apparatus;
transmitting, by the server controller, a set of treatment parameters associated with the light therapy treatment to the computing device; and
automatically initiating, by the computing device, the light therapy treatment at the light therapy apparatus associated with and remote from the computing device.

14. The method of claim 13, wherein the light therapy apparatus comprises:
a main chamber comprising a controller;
a first side chamber flexibly mounted to the main chamber, the first side chamber comprising a first power supply;
a second side chamber flexibly mounted to the main chamber, the second side chamber comprising a second power supply; and
a circuit board partially enclosed within the main chamber, the first side chamber and the second side chamber, the circuit board comprising:
a main board portion partially enclosed within the main chamber, the main board portion comprising a main array of light-emitting diodes (LEDs) and a controller mating member to engage with the controller;
a first board portion partially enclosed within the first side chamber, the first board portion comprising a first side array of LEDs and a first power mating member to engage with the first power supply;
a second board portion partially enclosed within the second side chamber, the second board portion comprising a second side array of LEDs and a second power mating member to engage with the second power supply;
a first linking member connecting the first board portion to the main board portion; and
a second linking member connecting the second board portion to the main controller portion.

15. The method of claim 13, wherein the injury position signal comprises a user identifier corresponding to a user with the injury and operating the server controller to define the light therapy treatment for the injury identified by the injury position signal comprises:
retrieving a user profile stored in a memory remote from the computing device for the user; and
defining the light therapy treatment based at least on a treatment history associated with the user profile.

16. The method of claim 15, wherein defining the light therapy treatment based at least on the treatment history associated with the user profile comprises:
identifying a prior injury similar to the injury; and
defining the light therapy treatment for the injury based on a recovery progress of the prior injury.

17. The method of claim 15, wherein defining the light therapy treatment based at least on the treatment history associated with the user profile comprises:
identifying an ongoing treatment for the injury;
determining a recovery progress of the injury based on the ongoing treatment; and
adjusting the ongoing treatment for the injury based on the recovery progress of the injury.

18. The method of claim 17, wherein determining the recovery progress of the injury based on the ongoing treatment comprises:
generating a progress assessment interface at the computing device requesting an assessment of the recovery progress from the user; and
receiving a recovery assessment signal from the computing device representing the recovery progress of the injury.

19. The method of claim 13 further comprising collecting patient data feedback to vary the treatment parameters of the light therapy apparatus.

20. The method of claim 13 further comprising:
prior to initiating the light therapy treatment at the light therapy apparatus, operating the computing device to detect a power supply level at the light therapy apparatus; and
in response to determining the power supply level is below a minimum power threshold, transmitting a power deficient notification to the computing device, otherwise, initiating the light therapy treatment at the light therapy apparatus.
